(19) United States
(12) Patent Application Publication
(10) Pub. No.: US 2004/0107592 A1
(43) Pub. Date: Jun. 10, 2004

(54) Ergonomics instrument kit

(76) Inventor: Monica V. Matlis, Yardley, PA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/315,552

(22) Filed: Dec. 10, 2002

(51) Int. Cl. A61B 5/103

(52) U.S. Cl. 33/512

(57) ABSTRACT

The present invention relates to an ergonomics instrument kit, and more particularly to a kit that includes the technical instruments necessary for the identification and evaluation of various ergonomics risks factors in the workplace. The ergonomics instrument kit comprising a plurality of Rulers for evaluating Occupational Posture by measuring the physical relationship between body parts or a plurality of Force Measurement Instruments for evaluating Occupational Force by measuring the amount of force exerted by a human performing an activity. The kit may also include combinations of Rulers and Force Measurement Instruments. Rulers may include instruments such as goniometers and bubble inclinometers. Force Measurement Instrument may include instrument such as pinch gauges, push-pull gauges, and dynamometers.

### FIELD OF THE INVENTION

The present invention relates to an ergonomics instrument kit, and more particularly to a kit that includes the technical instruments necessary for the identification and evaluation of various ergonomics risks factors in the workplace.

### BACKGROUND OF THE INVENTION

The term “ergonomics” is derived from two Greek words: “ergon”, meaning work and “nomos”, meaning principle or law. Together the terms means the Science of Work, that is, the use of Man's forces and faculties (human capabilities) in relationship to work demands. Accordingly, the term ergonomics today encompasses the scientific study of human beings in relation to their environment. Ergonomics is an approach that puts human needs and capabilities at the focus of designing technological systems, with the aim of ensuring that humans and technology work in complete harmony.

Today's business environment has become increasingly competitive—requiring higher production rates and advances in technology. As a result, jobs today can involve repetitive motions of body parts (such as the hands, wrists and arms), frequent lifting, carrying, and pushing or pulling loads without help from other workers or devices. In addition, workers are often required to work more than 8 hours per day, work at a quicker pace, and/or have tighter grips when using tools. These factors, especially when coupled with poor machine, tool, and workplace design, or the use of improper tools, create physical stress on the workers' bodies, which can lead to injury.

Medical professionals have long observed the correlation between occupational related activities and injuries of the soft tissues (muscles, tendons, ligaments, joints, and cartilage) and nervous system. These “disorders”, commonly referred to as musculoskeletal disorders (MSD's) or cumulative trauma disorders (CTD's), can affect nearly all tissues, including the nerves and tendon sheaths, and most frequently involve the arms and back. Occupational safety and health professional also refer to these disorders by a variety of terms, including cumulative trauma disorders, repeated trauma, repetitive stress injuries (RSI's), and occupational overexertion syndrome. Hereinafter, these “disorders” will be individually and collectively referred to as MSD's.

Recent ergonomics studies have clearly established a relationship between certain occupational activities and MSD's. This research has identified numerous conditions that are likely to cause MSD problems, including: exerting excessive force; excessive repetition of movements; awkward posture, or unsupported positions that stretch physical limits; static postures, or positions that workers must hold for long periods or time; motion, such as increased speed or acceleration when bending and twisting; compression, from grasping sharp edges, like tool handles; inadequate recovery time due to overtime, lack of breaks, and failure to vary tasks; and excessive vibration. Although these conditions may seem somewhat unrelated, there are two common elements that may contribute to the musculoskeletal problems—particularly “Occupational Posture” and “Occupational Force”.

Occupational Posture refers to the effort or work to perform a particular activity, and may be further classified into two categories: “static posture” and “repetitive posture” (also known as dynamic posture). Static posture refers to the effort or work to hold a particular position, particularly the musculoskeletal effort required to hold a particular position during an occupational activity. For example, when we sit and work at a computer, we must keep our head and torso upright. Depending upon the posture that we choose, or are required to exhibit by the physical limits of our work place, either small or great amounts of static posture will be required. Improper static postures, or positions that a worker must hold for long periods of time, can restrict blood flow and damage muscles. In addition, awkward postures or unsupported positions that stretch physical limits can compress nerves and irritate tendons.

Repetitive posture includes the effort or work to complete excessive repetition of movements by a worker, such as for example typing on a computer keyboard, opening doors or latches, turning knobs or levers, opening guards, reaching for products on an assembly line, loading conveyors, lifting objects, reaching for objects, repetitive bending of the wrist elbow, knee or ankle joints, etc. Repetitive motion can irritate tendons and increase pressure on the nerves.

Other factors, such as excessive vibration, or whole-body vibration can also contribute to nerve damage and muscle fatigue. These factors may also be classified as Occupational Posture elements that contribute to MSD's.

Occupational Force, on the other hand, refers to the amount of tension (or compression) a worker's muscles generate or experience during an occupational activity. Occupational Force can come in several forms. For example, tilting your head forward or backward from a neutral, vertical position increases the muscular tension (tension force) necessary to support your head. The movement can actually quadruple the amount of force acting on your lower neck vertebra. Other tensile forces, such as those caused by repetitive pulling and straining can injure the muscles and ligaments of the back. This condition may cause the back muscles, discs, and ligaments to become scarred and weakened, losing their ability to support the back. Other forces, such as compressive force and contact stresses can also have a detrimental effect on your body. Compression, for example, from grasping sharp edges, like tool hands, can concentrate force on small areas of the body. These compressive forces may reduce blood flow and nerve transmission, and damage tendons and tendon sheaths. In addition, increased speed or acceleration when bending or twisting, or excessive repetition of movements can increase the amount of force exerted on or by the body, irritating tendons and muscles and increasing pressure on nerves. Environmental conditions, such as working in cold temperatures may exacerbate these conditions by adversely affecting a worker's coordination and manual dexterity. These diminished conditions can cause a worker to use more force than necessary to perform a task.

MSD's not only take their toll on workers, but they can negatively affect a company's financial health and well-being. For example, it has been reported that MSD's account for 34 percent of all lost-workday injuries and illnesses. That translates to nearly 600,000 MSD's requiring time away from work every year. MSD's also account for $1 of every $3 spend for workers' compensation or $15 to $20 billion in workers' compensation costs annually. All factors considered, the total direct costs associated with MSD's accounts for as much as $50 billion annually.

Many solutions to ergonomics problems in the workplace are simple and inexpensive to implement. Sometimes just establishing an ergonomics program, including a job hazard analysis, and encouraging worker's to participate in the program may reduce worker incidence of MSD's. A job hazard analysis identifies problem jobs and risk factors associated with them. It may also identify jobs and work stations that are the source of greatest problems. By simply measuring the Occupational Posture and Occupational Force experienced by a worker, an employer may identify factors that can contribute to a worker's musculoskeletal disorders. By altering the way the worker conducts his or her job, such as by improving posture, allowing tasks modification (such as alternate activities), or providing tool and/or environment modifications (engineering controls), the employer may help control ergonomics risk factors and prevent MSD hazards.

There are other significant benefits to establishing an ergonomics program, including worker efficiency, worker productivity, improved equipment/workstation design, and safety and health in the work environment. Some examples of improvements motivated by ergonomics factors include: designing equipment and systems that are easier to use and less likely to lead to errors in operation; designing tasks and jobs so that they are effective and take human needs into account; designing equipment and work arrangements to improve working posture and ease the load on the body, thus reducing instances of MSD's; and designing the working environment, including lighting and heating, to suit the needs of the users and the tasks being performed.

In addition, a tremendous amount of attention has been paid to ergonomics on both the state and national level in recent years. Presently, the Occupational Safety and Health Administration (OSHA) is developing industry, or task-specific guidelines for a number of industries. These guidelines will be based on current incidence rates and available information about effective and feasible solutions. Once relevant standards have been established, OSHA will enforce the provisions of these standards through inspections for ergonomics hazards, and issue citations where appropriate.

Various instruments are sold that can measure occupational risk factors, such as those associated with Occupational Posture and Occupational Force experienced by employees in the workplace. However, workers come in many shapes and sizes, and occupational risk factors vary from work location to work location. Therefore, it can be appreciated that there exists a need for ergonomics instrument kits that include the technical instruments necessary to identify a wide variety of ergonomics risks, caused by both Occupational Posture and Occupational Force, in the workplace.

### SUMMARY OF THE INVENTION

The present invention relates to an ergonomics instrument kit that includes the technical instruments necessary for the identification and evaluation of various ergonomic risks factors in the workplace. In one embodiment of the invention, the ergonomics instrument kit comprises a plurality of instruments to assist in evaluating Occupational Posture.

Another embodiment of the invention comprises a plurality of force measurement instruments to assist in evaluating Occupational Force.

Another embodiment of the ergonomics instrument kit comprises one or more instruments to assist in evaluating Occupational Posture and one or more force measurement instruments to assist in evaluating Occupational Force.

Still another embodiment of the ergonomics instrument kit comprises a digit goniometer, a medium goniometer, a large goniometer, a bubble inclinometer, a pinch gauge, a dynamometer, and a push-pull gauge.

Another embodiment of the ergonomics instrument kit comprises a means for measuring Occupational Posture and a means for measuring Occupational Force, each individually and in combination.

### BRIEF DESCRIPTION OF THE DRAWINGS

FIG. 1 shows a perspective view of an ergonomics instrument kit according to one embodiment of the present invention.

FIG. 2A shows a front view of a digit goniometer according to one embodiment of the present invention.

FIG. 2B shows a perspective view of a digit goniometer in operation according to one embodiment of the present invention.

FIG. 3 shown a front view of a large goniometer according to one embodiment of the present invention.

FIG. 4A shows a front view of a medium goniometer according to one embodiment of the present invention.

FIG. 4B shows a perspective view of a medium goniometer in operation according to one embodiment of the present invention.

FIG. 5A shows a front view of a bubble inclinometer according to one embodiment of the present invention.

FIG. 5B shows a perspective view of a bubble inclinometer in operation according to one embodiment of the present invention.

FIG. 6A shows a perspective view of a dynamometer according to one embodiment of the present invention.

FIG. 6B shows a perspective view of a dynamometer in operation according to one embodiment of the present invention.

FIG. 7A shows a top view of a pinch gauge according to one embodiment of the present invention.

FIG. 7B shown a perspective view of a pinch gauge in operation according to one embodiment of the present invention.

FIG. 8A shows a front view of a push-pull gauge according to one embodiment of the present invention.

FIG. 8B shows a perspective view of a push-pull gauge in operation according to one embodiment of the present invention.

### DESCRIPTION OF THE PREFERRED EMBODIMENT

As will be described below, the invention relates to a kit comprising instruments for taking various ergonomics measurements, particularly instruments for assessing ergonomics risk experienced by workers in the workplace. The ergonomics kit contemplates, and may also include, other devices and instruments for measuring other occupational risk factors, such as, light, sound, temperatures (hot and cold), quick motions, compression or contact stresses, and vibrations. These other ergonomics instruments may include, for example, a light meter for measuring the amount of light at a work location, and an audio meter for measuring the sound or sound pressure level at a workplace, an accelerometer or vibration measuring device for measuring acceleration and vibration, a thermometer for measuring temperature, and camera or video equipment for visualizing contact stresses.

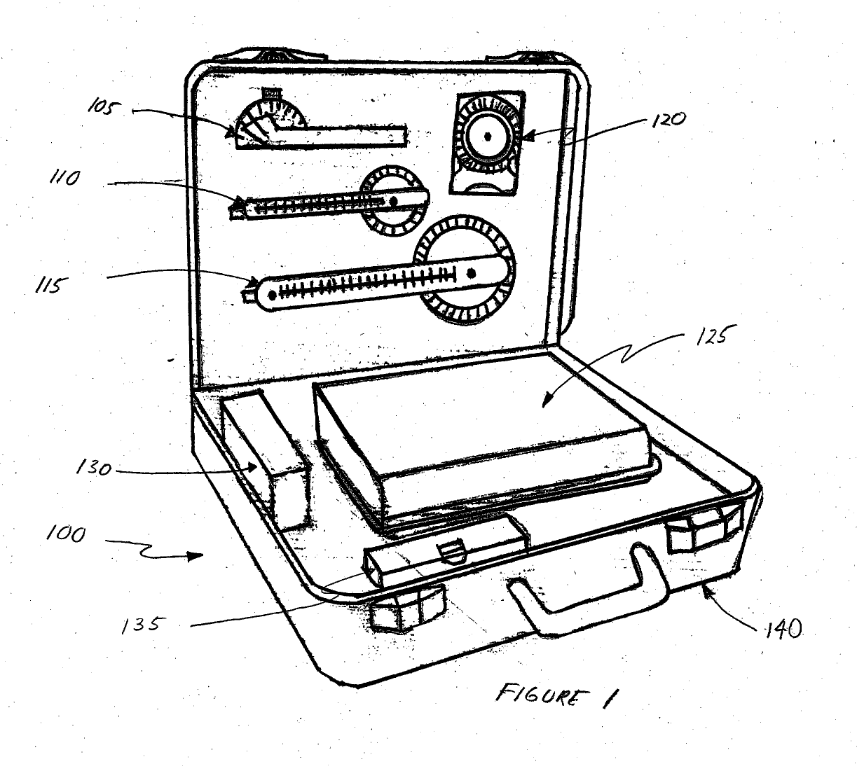

An ergonomics instrument kit 100 according to one embodiment of the present invention is illustrated in FIG. 1. Although the kit 100 shows numerous instruments for measuring Occupational Posture and Occupational Force, it is not meant to limit the scope of the invention. Many other combinations of these instruments and other ergonomics instruments are also contemplated as will be understood by one of ordinary skill in the art.

The instrument kit 100 is generally comprises two types of instruments: “Rulers” that measure the physical relationship between body parts, and thus assist with the ergonomics evaluation of Occupational Posture; and Force Measurement Instruments that measure the amount of Occupational Force a worker exerts or experiences performing an activity. As the term is used herein, Rulers include instruments for taking linear measurements, such as the distance between two body parts or between a body part and a work place device; and angle measuring devices for measuring the geometric relationship between two body parts or a body part and a work place device or surface.

Referring to FIG. 1, the Rulers used to evaluate Occupational Posture (static or repetitive motion) experienced by a worker include three different size goniometers (digit goniometer 105, medium goniometer 110, and large goniometer 115) and a bubble inclinometer 120.

The goniometers (105, 110, 115) generally measure the angular relationship, i.e. the movement or axis or motion and static range of motion of a worker's joint. The joint may be between body appendages, such as for example the fingers (knuckles), hands (wrists), arms (elbows and shoulders), legs (knees), feet (ankles) and toes (toe joints). In addition, the goniometers may also be used to measure the angular relationship of other body parts, including the neck (angular relationship of the head to the body) and hips (angular relationship of the torso to the legs). The goniometers (105, 110, 115) may also include a ruler or linear scale to allow a user to take linear distance measurements.

The angular relationship between articulating body parts is very important for an ergonomics evaluation. Recent studies have shown that work activities should be performed with the joints at about the mid-point of their range of movement. This applies particularly to the head, trunk and upper limbs.

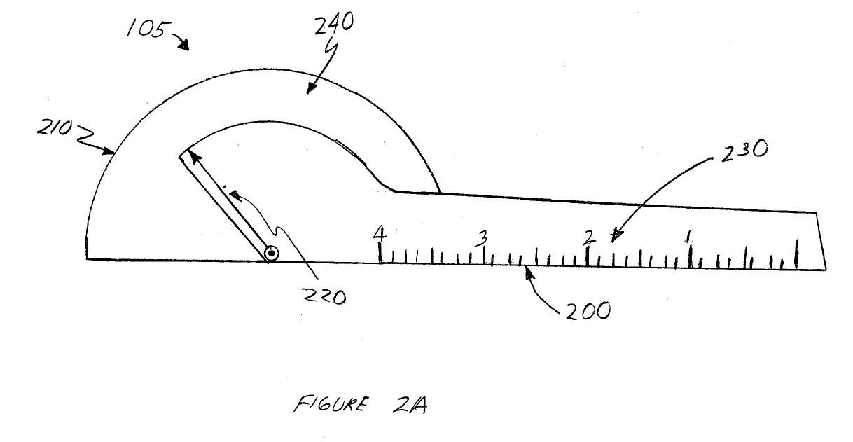

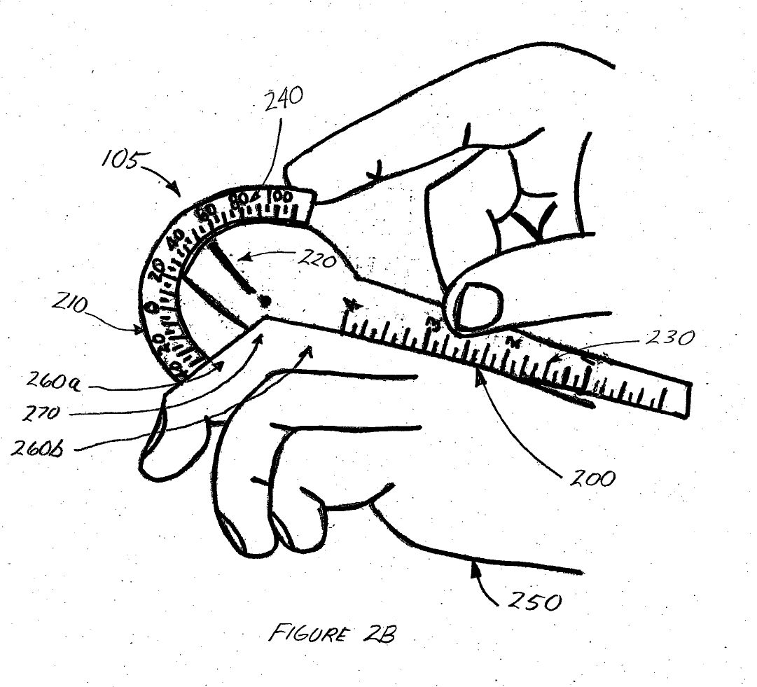

A digit goniometer 105 according to one embodiment of the present invention is shown in FIGS. 2A and 2B. The digit goniometer 105 is used to measure the angular relationship of the finger joints. As illustrated in FIG. 2A, the digit goniometer 105 comprises a base arm 200 and a measurement head 210. The base arm 200 includes a reference line 220 for reading the joint angle directly on an angular scale 240 integrated into measurement head 210. The base arm 200 also includes a 4-inch (10 centimeter) linear scale 230 for taking linear distance measurements. In the illustrated embodiment, the angular scale 240 is capable of measuring angles from 40 degrees hyperextension to 110 degrees flexion in 5-degree increments. As shown in FIG. 2B, by placing the digit goniometer 105 on the dorsal side of a hand 250, a workers finger can be rotated to read the angular relationship between various segments of the finger 260a and 260b on each side of a finger joint 270 or knuckle.

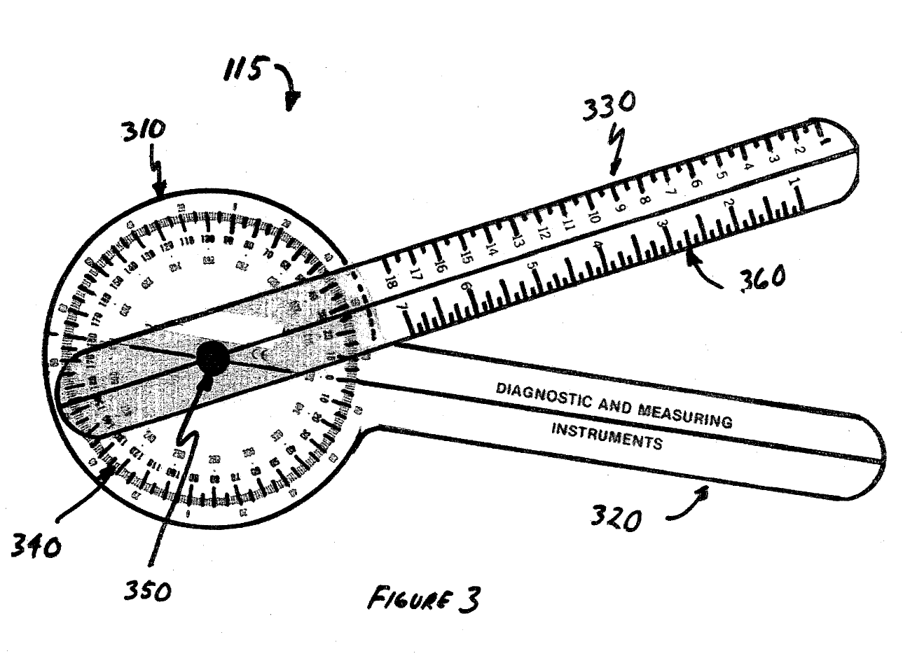

FIG. 3 illustrates a large goniometer 115 according to one embodiment of the present invention. This instrument may be used to measure the angular relationship, i.e. the movement axis or static/dynamic range of motion of joints connecting the larger appendages, such as, for example, the legs, arms and torso. Large goniometers, such as large goniometer 115, are generally considered to include goniometers having lengths between 8 inches to 15 inches, and preferably having lengths of about 12 inches. The large goniometer 115 comprises two main members, a base leg 320 having a protractor head 310 integrated therein, and an articulating leg 330. The base leg 320 and articulating leg 330 are pivotally connected at pivot point 350. This configuration allows the articulating leg 330 and base leg 320 to be angularly rotated with respect to one another.

The protractor head 310 has at least one angle scale 340 for reading the angular measurement or relationship between the base leg 320 and articulating leg 330. In a preferred embodiment, the protractor head 310 has three angle scales 340 to measure the angular relationship; an outer scale, middle scale and inner scale. The outer scale is divided into four segments, each measuring angles between 0 and 90 degrees in 1-degree increments. The middle scale is divided into two segments, each measuring angles between 0 and 180 degrees in 1-degree increments. Finally, the inner scale is divided into two segments, each segment measuring angles between 180 and 360 degrees in 1-degree increments.

One or both of the legs 320, 330 may have a linear scale 360 for measuring linear distance. In the embodiment illustrated, articulating leg 330 comprises a linear scale calibrated to read linear distance in both standard and metric units.

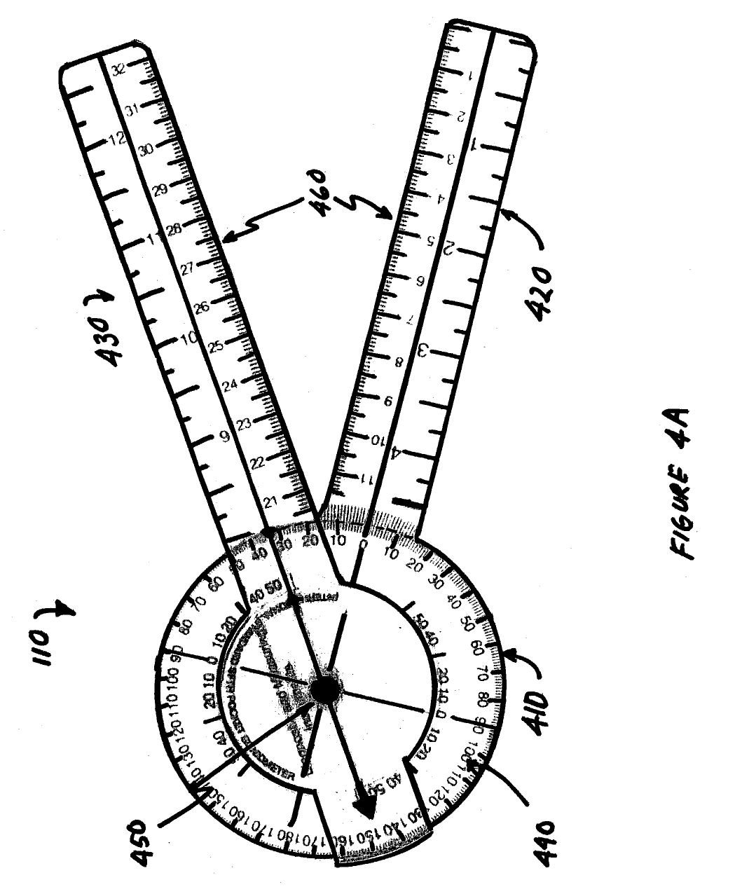

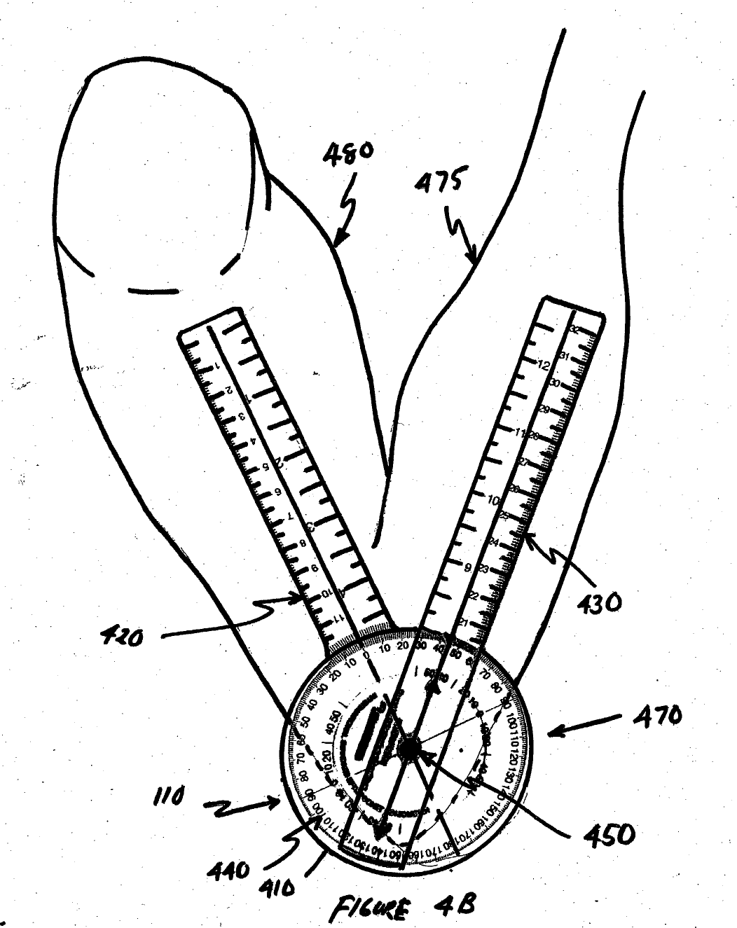

A medium goniometer 110 according to one embodiment of the present invention is illustrated in FIG. 4A. Similar to the large goniometer 115, the medium goniometer 110 may be used to measure the angular relationship of joints connecting small to medium sized appendages, such as, for example, the arms, hands and feet. Medium goniometers, such as medium goniometer 110, are generally considered to include goniometers having lengths between 6 inches and 10 inches, and preferably 8 inches in length. The medium goniometer 400 also comprises two main members, a base leg 420 having a protractor head 410 integrated therein, and an articulating leg 430. The base leg 420 and articulating leg 430 are pivotally connected at pivot point 450. This configuration allows the articulating leg 430 and base leg 420 to be angularly rotated with respect to one another. In addition, one or both of the legs 420, 430 may have a linear scale 460 for measuring linear distance. In the embodiment illustrated, articulating leg 430 and base leg 420 each include a linear scale calibrated to read linear distance in both standard and metric units.

The protractor head 410 also has at least one angle scale 440 for reading the angular measurement or relationship between the base leg 420 and the articulating leg 430. In a preferred embodiment, the protractor head 410 has two angle scales 440, an outer scale and an inner scale, to measure the angular relationship. The outer scale is divided into two segments, each measuring angles between 0 and 180 degrees in 1-degree increments. Similarly, the inner scale is divided into four segments, each measuring angles between 0 and 60 degrees in 10-degree increments.

The operation of the medium and large goniometer 110, 115 are very similar. FIG. 4B shows a perspective view of the medium goniometer 110 being used to measure the range of motion of an elbow joint 470, i.e. the forearm 475 in relation to the upper arm 480. The worker is first instructed to bend the arm at the elbow 470 into the full flexion position (as illustrated). The appropriate sized goniometer, in this case medium goniometer 110, is placed with the base leg 420 parallel to the upper arm 480, and the pivot point 450 concentric with the elbow 470 axis of rotation. The articulating leg 430 of the goniometer 110 is then rotated about pivot point 450 until it is parallel with the forearm 475. The minimum angle for the range of motion can then be read off angle scale 440 on protractor head 410. The worker is then instructed to rotate the arm about the elbow joint 470 to the full extension position (not shown), and the maximum angle measurement is read in a similar fashion as described above.

Although not illustrated, the ergonomics instrument kit 100 may also include a small goniometer. The small goniometer is generally less than 6 inches in length and used to measure small appendages, such as for example the arms, hands, and feet. The small goniometer may also be used to measure the angular relationship between the head, neck and shoulders.

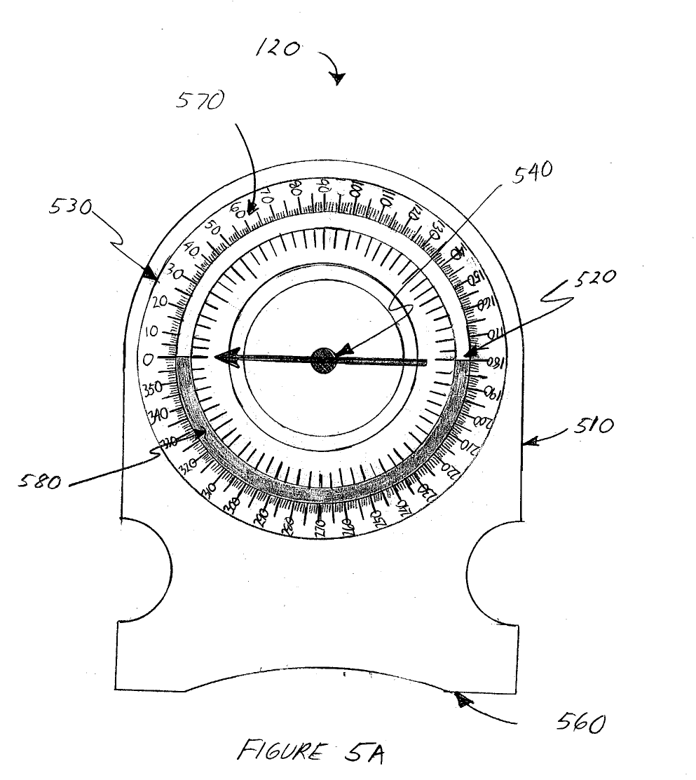

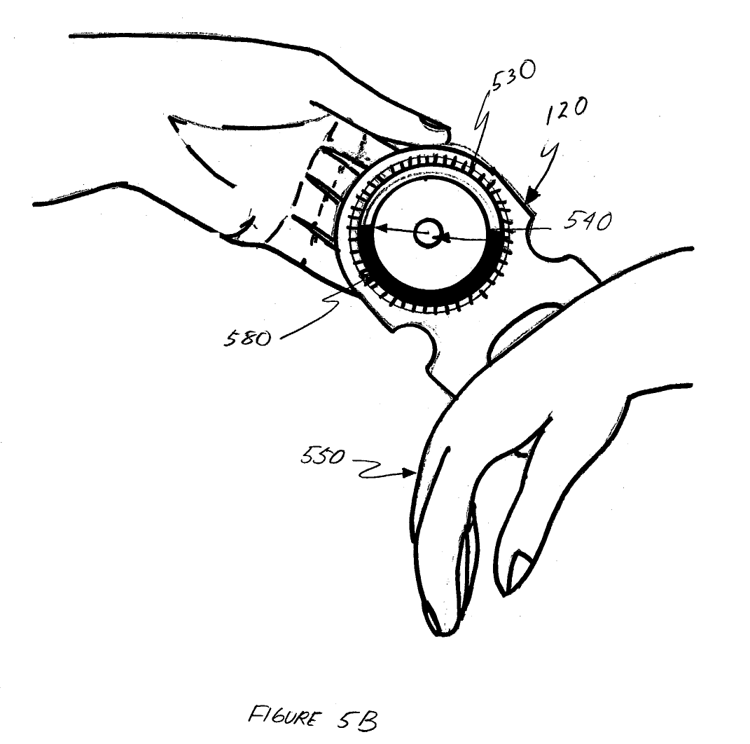

A bubble inclinometer 120 is shown in FIGS. 5A and 5B. The bubble inclinometer 120 may be used to measure angular position and range of motion of small surfaces, like a hand 550, where angles are not easily obtained with conventional goniometers. The bubble inclinometer 120 may also be used to measure flexion and extension; abduction and adduction; and rotation in the neck, shoulder, elbow, wrist, hip, knee, ankle and spine.

In a preferred embodiment of the invention, the bubble inclinometer 120 comprises a housing 510 holding a fluid channel 520 and a base 560. The inclinometer 120 also has a dial 530 rotateably mounted to the housing 510 about a pivot point 540. The dial 530 is provided with a scale 570 capable of reading angles from 0 to 360 degrees in 1-degree increments. A colored fluid 580 fills the channel 520 to assist the operator in reading the angular relationship when taking a reading. The fluid 530 is of a viscosity sufficient to permit a fast accurate reading without waiting for oscillations to dampen the fluid movement.

One measurement taken by the bubble inclinometer 500 is the range of motion of a body part, such as for example, the hand 550 illustrated in FIG. 5B. The bubble inclinometer 500 is first placed on the dorsal side of hand 550 with the hand 550 in the complete flexion position. The dial 530 is then rotated about pivot point 540 until the 0-degree mark (arrow) is aligned with the colored fluid 580 in fluid channel 520. The hand 550 is then moved to the full extension position. The range of motion (in degrees) can then be read directly off the dial 530, i.e. the new point on the scale (degree) that is aligned with the colored fluid 580 in fluid channel 520.

The above-disclosed instruments describe the Rulers used in a preferred embodiment of the present invention used to measure Occupational Posture. Many other instruments may also be included, or substituted, in other embodiments. These other instruments may include, for example: a measuring tape; other variations or sizes of goniometers and inclinometers; a Cervical Range of Motion Instrument (CROM) for measuring suboccipital flexion and extension, cervical flexion and extension, lateral flexion, and rotation, and forward head motion; a Back Range of Motion Instrument (BROM) for measuring flexion, extension, rotation, and lateral flexion of the back, and pelvic tilt; a Biplane Goniomoeter to access ankle dorsiflexion; a two point discriminator; or a Sit and Reach Flexibility Tester to measure hip and lower back flexibility. Still other Rulers may be used as would be understood by one of skill in the art.

The second type of instruments shown in FIG. 1 are Force Measurement Instruments. As the term implies, Force Measurement Instruments are use to measure the amount of Occupational Force (tension or compression) a worker's muscles generate or experience during particular activities. Referring again to FIG. 1, the ergonomics instrument kit comprises the following Force Measurement Instruments in one embodiment of the invention: a dynamometer 125; a pinch gauge 130; and a push-pull gauge 135.

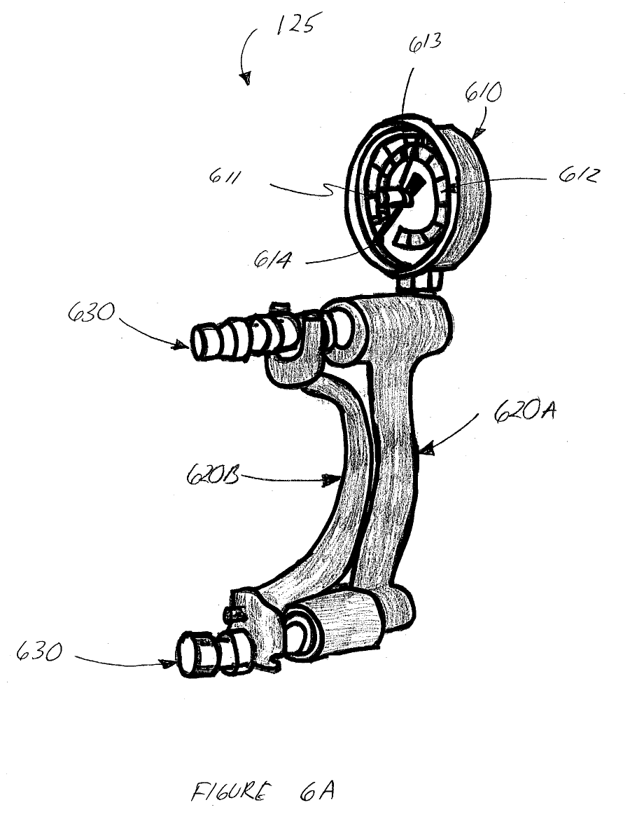

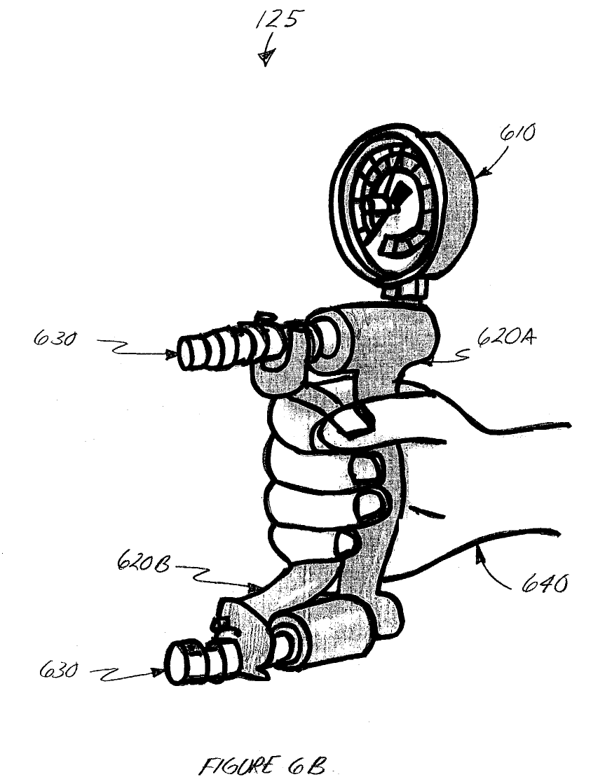

A perspective view of the dynamometer 125 according to one embodiment of the present invention is shown in FIGS. 6A and 6B. The dynamometer 125 comprises a first and a second handle 620A, 620B respectively, adjustment studs 630, and gauge 610.

Gauge 610 includes an indicator needle 614 and scale 612 for reading hand grip strength. In a preferred embodiment, the scale 612 records in both pounds and kilograms. Preferably, the gauge 610 also includes a maximum output indicator 613 for indicating the maximum grip strength achieve during operation, and a reset knob 611 for resetting the maximum output indicator 613 before each use. The gauge 610 may also include a zero adjustment pin so that the “zero-range” of the needle indicator may be set if necessary.

The studs 630 comprise a plurality of adjustment detents for allowing the handle 620B to be adjustably attached to the studs 630. The studs 630 are also slidably connected to handle 620A such that the inward force or movement of the studs 630 into handle 620A is recorded on gauge 610.

The operation of the dynamometer 125 is illustrated in FIG. 6B. Prior to operation, the handle 620B of dynamometer 125 should be adjusted on studs 630 so that a comfortable grip for the worker is achieved. The maximum output indicator 613 should also be set to zero. The worker may then squeeze the handles 620A, 620B together with maximum force, and the reading on the scale 612 noted.

Another force measuring device (Force Measurement Instrument) contemplated by the ergonomics instrument kit illustrated in FIG. 1 is pinch gauge 130. The pinch gauge 130 is used for measuring the force of thumb-finger prehension (pinch force). Pinch force may be defined as the force associated with the act of taking hold, seizing, or grasping between a thumb and finger. The pinch gauge 130 is capable of measuring prehension using several different methodologies, including: the Tip Pinch (thumb tip to index fingertip); the Key Pinch (thumb pad to lateral aspect of middle phalanx of the index finger); and the Palmar Pinch (thumb pad to pads of the index and middle fingers).

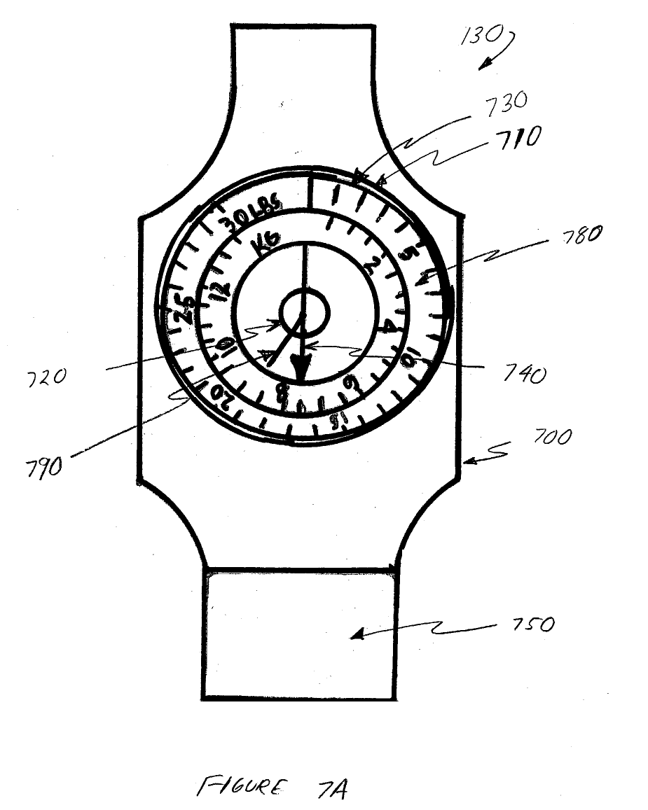

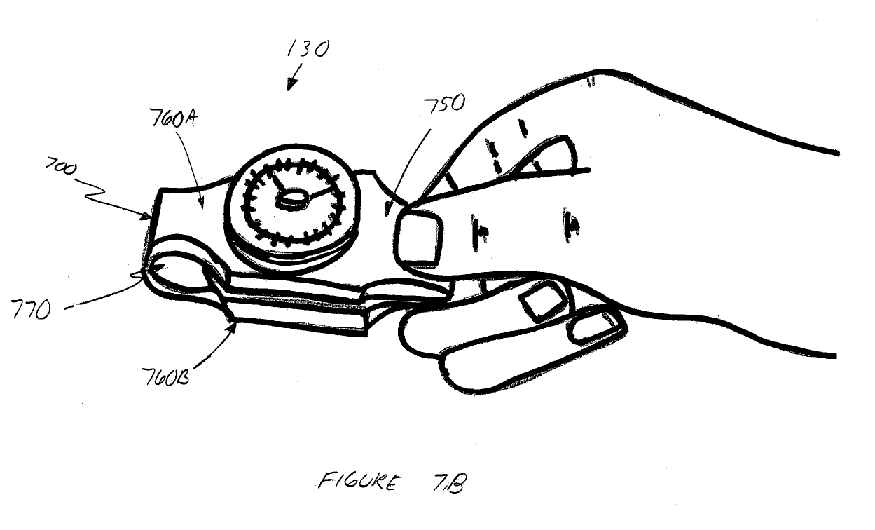

FIGS. 7A and 7B show front and perspective views of pinch gauge 130 according to one embodiment of the present invention. The pinch gauge 130 comprises a body 700 having first and second substantially parallel sides 760A, 760B respectively, each having a pinch pad 750 incorporated therein. The pinch pads 750 provide a proper location for finger placement during thumb-finger prehension measurement. The sides 760A, 760B are connected to each other by flex joint 770. Flex joint 770 is sufficiently deformable to allow sides 760A and 760B to be urged closer to each other when pinch force is applied. The resistance to this deformation inherent in flex joint 770 is directly related to the force measurement of the pinch gauge 130.

Pinch gauge 130 also comprises a dial indicator to measure the amount of deformation between sides 760A and 760B when the force is applied by the fingers to the pads 750. As previously described, since the resistance to deformation is known, the amount of deformation measured by the dial indicator is directly related to the amount of pinch force generated by the fingers squeezing the pinch gauge 130.

The dial indicator comprises a gauge 730 incorporating a dial scale 780. In a preferred embodiment, the dial scale 780 is capable of reading the force in both English and Metric units. The gauge 730 further comprises a knurled ring 710 on the exterior surface of the gauge 730 body to allow the gauge housing to be grasped and rotated. This allows for adjustment of the zero point on the gauge. To read the pinch force generated by the fingers during operation, the gauge 730 has an indicator arrow 740. When the gauge is operated, the pinch force readings can be read directly off the scale 780. To assist the operator, a preferred pinch gauge also includes a maximum force indicator hand 790 that identifies the greatest pinch force exerted on the pinch gauge 130. The maximum force indicator hand 790 is affixed to adjustment knob 720 rotably attached to the gauge 730. The maximum force indicator hand 790 may be adjusted and reset by rotating the adjustment knob 720 to the desired position.

To operate the pinch gauge 130 the operator grasps the knurled ring 710 on the gauge 130 body, and rotates the gauge 730 body until the zero on the scale 780 face is directly under the indicator arrow 740. To adjust the maximum force indicator hand 790, the operator rotates the adjustment knob 720 in a counterclockwise direction until the hand 790 rests against the indicator arrow 740 (at the zero position on the scale 780). When pinch force is measured, the maximum force indicator hand 790 will remain at the maximum reading until reset by rotating adjustment knob 720. The maximum force indicator hand 790 should be reset to zero prior to each force measurement. The worker may then grasp the pinch gauge 130 at the pad 750 using the digits dictated by the desired methodology and squeeze the pads 750 towards each other. Pinch force (prehension) may then be read directly off scale 780.

Another Force Measurement Instrument contemplated by the ergonomics instrument kit illustrated in FIG. 1 is push-pull gauge 135. The push-pull gauge 135 accurately measures the amount of force it takes to accomplish a task, such as, for example pushing or pulling bins, product carts, dumpsters, etc. A preferred embodiment of this instrument is capable of measuring both compression and tension forces.

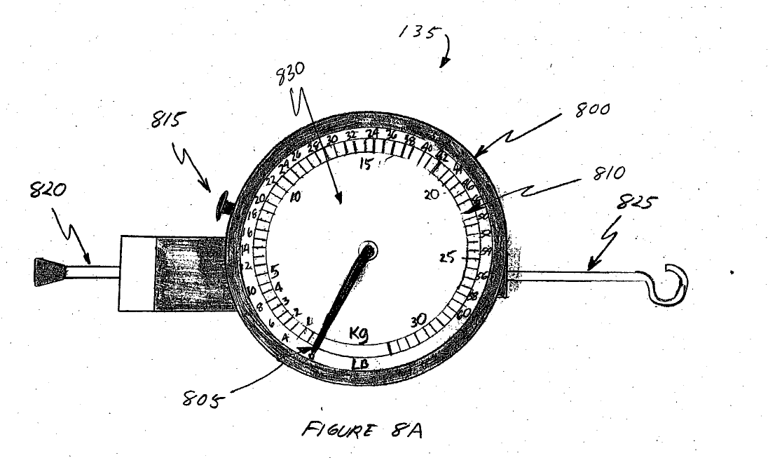

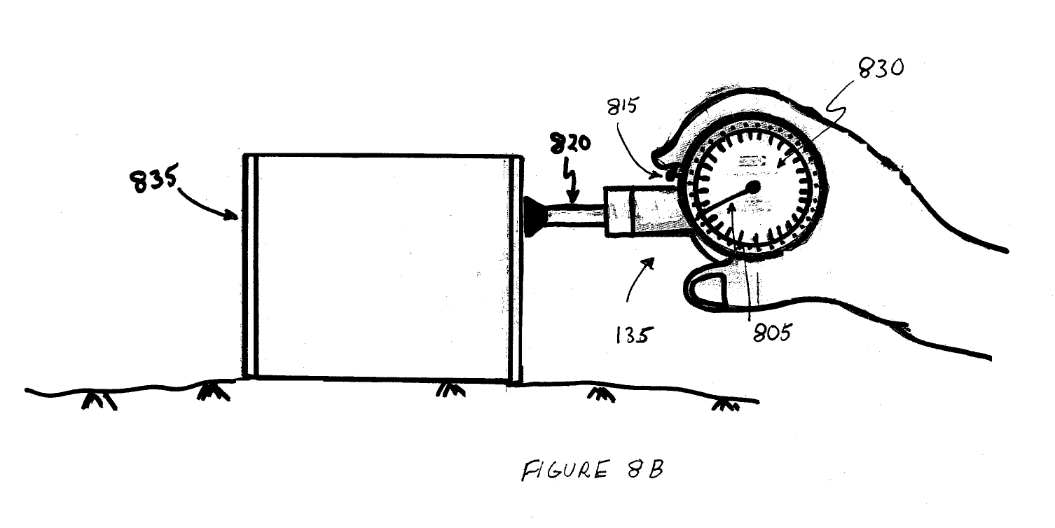

FIG. 8A shows a front view of the push-pull gauge 135 according to one embodiment of the present invention. The push-pull gauge 135 includes a housing 800 that houses a force dial 830. The force dial has a scale 810 used to read the amount of force measured by the gauge 135. In a preferred embodiment, the scale 810 has dual graduations for measurement in both English units (pounds and ounces) and Metric units (kilograms and grams). The push-pull gauge 135 also includes plunger 820 and/or tension hook 825. These items may be permanently attached to the push-pull gauge 135, but are preferably removable.

The maximum or peak force measured by the push-pull gauge 135 is indicated by a pointer 805. Preferably, the peak reading is saved by the pointer 805 remaining in the position of maximum applied force after the force is removed. This allows an operator to easily read the maximum force against the scale 810 on force dial 800. A reset button 815 resets the pointer 805 to the zero force position so that additional readings can be taken.

To measure the compressive force to accomplish a task, for example, the force to push a bin 835, a plunger 820 is attached to the push-pull gauge 135 as shown in FIG. 8B. When measuring compression, it is advantageous to remove tension hook 825. An operator first resets the pointer 805 to the “at rest” position by pushing the reset button 815. The operator may then contact the end of the plunger 820 to the bin 835 and apply force until the bin moves. It is important that the forces be applied to the plunger 820 in an axial manner. Applying a load at an angle can cause an error in readings and possibly damage the gauge. The maximum force to accomplish the task is indicated by the pointer 805 position on the dial 830, i.e. number on the scale 810 pointed to by pointer 805.

The tensile force to accomplish a task, for example, the force to pull a wheeled cart, can be performed in a similar manner. To measure tension (tensile force) the tension hook 825 is attached to push-pull gauge 135. After the operator resets the pointer 805 to the at rest position, the tension hook 825 may be attached or “hooked” to the cart, and a tension force asserted until the cart moves. The maximum force to accomplish the task is indicated by the pointer 805 position on the dial 830, i.e. number on the scale 810 pointed to by pointer 805.

The above-disclosed instruments describe the Force Measurement Instruments used in a preferred embodiment of the present invention to measure Occupational Force. Many other devices may also be included, or substituted, in other embodiments. These other Force Measurement Instruments may include, for example: a weight scale; a bulb squeeze dynamometer; or a back-leg-chest dynamometer. Still other Force Measurement Instrument may be used as would be understood by one of skill in the art.

While a number of variations of the invention have been shown and described in detail, other modifications and methods of use contemplated within the scope of this invention will be readily apparent to those of skill in the art based upon this disclosure. It is contemplated that various combinations or sub-combinations of the specific embodiments may be made and still fall within the scope of the invention. For example, an ergonomics instrument kit may include only a plurality of Ruler instruments, or only a plurality of Force Measurement Instruments. In addition, the ergonomics instrument kit may include other combinations of the illustrated instruments, or combinations of other instruments to measure or assist in the evaluation of Occupational Posture and Occupational Force. Accordingly, it should be understood that various applications, modifications and substitutions may be made of equivalents without departing from the spirit of the invention or the scope of the following claims.

What is claimed is:

1. An ergonomics instrument kit comprising a plurality of instruments to assist in evaluating Occupational Posture.

2. The ergonomics instrument kit of claim 1 wherein the instruments comprise a goniometer.

3. The ergonomics instrument kit of claim 2 wherein the goniometer is a digit goniometer.

4. The ergonomics instrument kit of claim 2 wherein the goniometer is a large goniometer.

5. The ergonomics instrument kit of claim 2 wherein the goniometer is a medium goniometer.

6. The ergonomics instrument kit of claim 2 wherein the goniometer is a small goniometer.

7. The ergonomics instrument kit of claim 1 wherein the instruments comprise a bubble inclinometer.

8. An ergonomics instrument kit comprising a plurality of instruments to assist in evaluating Occupational Force.

9. The ergonomics instrument kit of claim 8 wherein the instruments comprise a push-pull gauge.

10. The ergonomics instrument kit of claim 8 wherein the instruments comprise a pinch gauge.

11. The ergonomics instrument kit of claim 8 wherein the instruments comprise a dynamometer.

12. An ergonomics instrument kit comprising:
one or more Rulers to assist in evaluating Occupational Posture; and
one or move Force Measurement Instruments to assist in evaluating Occupational Force.

13. The ergonomics instrument kit of claim 12 wherein the Rulers comprise a goniometer.

14. The ergonomics instrument kit of claim 13 wherein the goniometer is a digit goniometer.

15. The ergonomics instrument kit of claim 13 wherein the goniometer is a large goniometer.

16. The ergonomics instrument kit of claim 13 wherein the goniometer is a medium goniometer.

17. The ergonomics instrument kit of claim 13 wherein the goniometer is a small goniometer.

18. The ergonomics instrument kit of claim 12 wherein the Rulers comprise a bubble inclinometer.

19. The ergonomics instrument kit of claim 12 wherein the Force Measurement Instruments comprise a push-pull gauge.

20. The ergonomics instrument kit of claim 12 wherein the Force Measurement Instruments comprise a pinch gauge.

21. The ergonomics instrument kit of claim 12 wherein the Force Measurement Instruments comprise a dynamometer.

22. An ergonomics instrument kit comprising:
a digit goniometer;
a medium goniometer;
a large goniometer;
a bubble inclinometer;
a pinch gauge;
a dynamometer; and
a push-pull gauge.

23. An ergonomics instrument kit comprising:
a means for measuring Occupational Posture; and
a means for measuring Occupational Force.

24. An ergonomics instrument kit comprising a means for measuring Occupational Posture.

25. An ergonomics instrument kit comprising a means for measuring Occupational Force.
